(19) United States
(12) Patent Application Publication
(10) Pub. No.: US 2014/0245866 A1
(43) Pub. Date: Sep. 4, 2014

(54) DEVICES AND METHODS FOR SANITIZING WRITING IMPLEMENTS

(71) Applicants: John Hadlock, Lexington, KY (US); Lee Gregg, Versailles, KY (US)

(72) Inventors: John Hadlock, Lexington, KY (US); Lee Gregg, Versailles, KY (US)

(21) Appl. No.: 14/192,103

(22) Filed: Feb. 27, 2014

Related U.S. Application Data
(60) Provisional application No. 61/771,114, filed on Mar. 1, 2013.

(51) Int. Cl. A61L 2/10; B43K 23/02

(52) U.S. Cl. CPC A61L 2/10; B43K 23/02
USPC 81/9.2; 250/455.11

(57) ABSTRACT

The present invention provides devices for disinfecting, sanitizing, etc., one or more writing implements using high energy source(s) or lamp(s), such as ultraviolet (UV) lamp(s), to irradiate the writing implement(s) placed within the device. The writing implement(s) may be placed inside an inner chamber surrounded by two or more portions of the device, the inner chamber receiving the writing implement(s) through a top opening and releasing or ejecting the writing implement(s) through a bottom opening. A holding mechanism present inside the device may hold the writing implement(s) until their release, such as in response to an input via a sensor and/or after a predetermined period of time. A device may instead have a receiving area or cavity that is irradiated by a high energy source(s) or lamp(s), which may be enclosed by a lid or cover. Writing implements having one or more separating fins, etc., are further provided.

### CROSS-REFERENCE TO RELATED APPLICATIONS

This application claims priority benefit to U.S. Provisional Patent Application No. 61/771,114, filed Mar. 1, 2013 and having the same title, the entire contents and disclosures of which is incorporated herein by reference.

### BACKGROUND

1. Field of the Invention

The present invention relates to devices and methods for cleaning or sanitizing writing implements between uses.

2. Related Art

Writing implements are often used repeatedly in a variety of public places including businesses, retail stores, health care facilities and other establishments for entering information and providing signatures either electronically or on paper, such as for authorizations, transactions or purchases. Given the number of times these writing implements can be handled and reused by different people over time, there is a high likelihood and risk that they may become contaminated and spread bacteria, viruses and other infectious agents that cause colds, flus, etc., especially in health care facilities where people may already be sick. The risk of infection can be a serious threat especially to some people including the elderly and immunocompromised individuals.

One solution might be to discard or throw away writing implements before their usable life has expired to reduce the possibility of spreading infectious agents. However, this approach is costly, wasteful and environmentally unfriendly. Another approach might be to periodically wipe or cleanse the writing implements with a sanitizing or disinfecting solution or wipe, but this approach would be time consuming and may result in an undesirable wet or sticky film or residue being left on the writing implement. Another drawback to both of these approaches is that they require someone to be responsible and mindful about discarding or cleaning the implement(s), which may also be vulnerable to oversight.

What is needed in the art is a new method and apparatus for cleaning or sanitizing writing implements between uses that avoids waste, does not require cleaning by hand or too much time or attention of personnel, and does not leave a film or residue on the writing implement that would be undesirable to subsequent users.

### SUMMARY

According to a first broad aspect of the present invention, a device is provided for disinfecting one or more writing implements comprising: two or more portions surrounding an inner chamber, the two or more portions comprising a front portion and a back portion; and at least one high energy source or bulb for directing disinfecting light toward the inner chamber of the device, each high energy source or bulb being positioned within one of the portions of the device, wherein the inner chamber has a top opening and a bottom opening, and wherein the horizontal cross-sectional shape of the inner chamber has an elongated width dimension and a depth dimension, the width dimension of the inner chamber being at least four times greater than the depth dimension of the inner chamber.

According to a second broad aspect of the present invention, a device for disinfecting a writing implement is provided comprising: a cradle portion having a receiving area, the receiving area having a length dimension and a depth dimension, the length dimension of the receiving area being at least four times greater than the depth dimension of the receiving area; at least one high energy source or bulb for directing disinfecting light toward the receiving area of the device; and a cover or lid, the cover or lid enclosing the space above the receiving area of the device when the cover or lid is in a closed position relative to the cradle portion of the device.

According to a third broad aspect of the present invention, a writing implement having elongated dimensions with a first longitudinal end and a second longitudinal, the first and second longitudinal ends being on opposite ends of the writing implement, the writing implement comprising: a plurality of outer fins including a first separating fin and a second separating fin, the first separating fin being closer to the first longitudinal end of the writing implement and the second separating fin being closer to the second longitudinal end of the writing implement.

These and other aspects of the present invention will become apparent to those skilled in the art after reading the following description and claims with the accompanying drawings.

### BRIEF DESCRIPTION OF THE DRAWINGS

The accompanying drawings, which are incorporated herein and constitute part of this specification, illustrate exemplary embodiments of the invention, and, together with the detailed description herein, serve to explain features of the present invention.

FIG. 1A is a perspective view of a sanitizing or disinfecting device for writing implements according to an embodiment of the present invention;

FIG. 1B is a cross-sectional view of the device in FIG. 1A according to an embodiment of the present invention from perspective of plane 1B;

FIG. 1C is a cross-sectional view of the device in FIG. 1A according to another embodiment of the present invention;

FIG. 2A is a perspective view of another sanitizing or disinfecting device for a writing implement or stylus according to an embodiment of the present invention;

FIG. 2B is a side view of the device in FIG. 2A according to an embodiment of the present invention;

FIG. 3 is a perspective view of another device for sanitizing or disinfecting a writing implement or stylus according to an embodiment of the present invention that attaches to an associated electronic device;

FIG. 4A is a perspective view of another sanitizing or disinfecting device for writing implements according to an embodiment of the present invention;

FIG. 4B is a top view of the device in FIG. 4A according to an embodiment of the present invention;

FIG. 4C is a cross-sectional view of the device in FIG. 4A according to an embodiment of the present invention from the perspective of plane 4C;

FIG. 5A is a perspective view of a cradle-like device for sanitizing or disinfecting a writing implement according to an embodiment of the present invention;

FIG. 5B is a cross-sectional view of the device in FIG. 5A according to an embodiment of the present invention from the perspective of plane 5B;

FIG. 5C is a longitudinal cross-sectional view of the device in FIG. 5A according to an embodiment of the present invention; and

FIG. 6 is a perspective view of a specialized writing implement according to an embodiment of the present invention.

### DETAILED DESCRIPTION

According to broad aspects of the present invention, devices and apparatuses are provided for disinfecting, cleaning, sanitizing, etc., one or more writing implements. An apparatus of the present invention may be used to disinfect, etc., a writing implement between uses of that implement without the use of cleaning solutions or other substances. An apparatus of the present invention will generally include an interior or inner chamber that may be mostly or fully enclosed by portions of the device and be designed to receive and/or produce high energy electromagnetic energy or light, such as ultraviolet (UV) light, to irradiate the interior or inner chamber of the apparatus and thus disinfect, etc., at least the outer surface(s) or portion(s) of the writing implement(s) placed, inserted, deposited, etc., therein. Ultraviolet or UV light includes electromagnetic energy or light having a wavelength within an approximate range of about 10 to 400 nm. Such UV light may more particularly include those wavelengths and intensities that have an anti-microbial and/or an anti-bacterial effect on exposed surfaces.

For the purpose of the present invention, the term “writing implement” refers to any instrument or implement that may be used to create a writing, marking or signature either electronically or on paper and may include any pencil, pen, stylus, highlighter, brush, etc. Such a “writing implement” may produce an image, signature, data entry, marking or lines on a surface by physically depositing or leaving behind an ink, pigment, lead, chalk or other substance, or by depressing a surface, such as an electronic surface that senses pressure, including an electronic writing or signature pad surface. A writing implement may include any implement or instrument commonly used at a retail point of sale, checkout counter, health care front desk or the like. Typically, a writing implement may have an elongated shape with the elongated sides of the implement being grasped by the hand of a user. The elongated writing implement generally has two longitudinal ends with one of those longitudinal ends being the writing end that contacts a surface to impart the electronic input or deposited marking, etc., of the writing implement.

According to embodiments of the present invention, an apparatus is provided having an inner enclosure or chamber defined and surrounded by a front, back and side portions, the inner enclosure or chamber being configured for receiving, containing and/or holding one or more writing implement(s). Each of the front, back and side portions of a device may comprise side wall(s), compartment(s), etc., and the inner walls of these portions may define the interior or inner chamber that receives the writing implement(s). A space or compartment may exist between and be defined by the inner and outer walls of each of these portions or compartments, which may each contain one or more high energy sources, such as UV lamps, for irradiating the writing implement(s). According to these embodiments, the inner wall(s) of one or more of the portions or compartments containing one or more high energy sources or lamps may be made of any suitable transparent material, such as glass, plastic, etc., to allow the energy or light created by the source(s) or lamp(s) to be transmitted through the inner wall(s) of these portion(s) to irradiate the writing implement(s) inside the chamber. According to some embodiments, one or both of the front and back portions or compartments may preferably contain one or more of these high energy sources or lamps. However, even though it may be more common or preferred for the front and/or back portions or compartments to contain one or more high energy sources or lamps, the side portion(s) may also or instead contain one or more high energy sources or lamps.

To contain and/or concentrate the electromagnetic energy waves or light (from the one or more high energy sources or lamps) inside the apparatus and chamber and direct that energy or light toward the writing implement(s), a reflective material may be present on the inner surfaces of the compartment(s) containing the one or more energy sources or lamps (other than the transparent inner wall) to redirect or reflect the energy or light toward the interior chamber. For example, the reflective material may be present on the inner surface of the respective outer wall(s) of a portion or compartment of the apparatus. The front, back and/or side portion(s) of the apparatus that do not contain an energy source or lamp may or may not have a compartment. If a front, back and/or side portion does not contain a compartment, then an inner (or only) wall of such portion may be non-transparent and/or may (or may not) have a reflective surface or material that faces toward the chamber. If these portions do happen to have a compartment defined by an inner wall and an outer wall but without any energy source or lamp inside the compartment, then the inner wall may be transparent or non-transparent, and/or the inner wall or outer wall of that portion of the device may have a reflective surface or material. One or more of the front, back and/or side portions of the apparatus may also lack an inner wall, and any energy or light generated by a high energy source or lamp that may be present within any such portion(s) of the apparatus may travel directly toward the writing implement(s) in the chamber without passing through any transparent inner wall. In these cases, that/those portion(s) of the device may have a cavity for housing the lamp/source that is continuous with the chamber.

According to aspects of the present invention, a mechanism is provided for releasing a writing implement from within the apparatus or device in response to an input from a user. While this input may be carried out by the user mechanically inputting the “command” or “request” to release the writing implement from within the device, such as by pulling a lever, pushing a button, etc., it is more preferable that this input be “hands-free.” According to these embodiments, a motion or light sensor (perhaps similar to sensors used in public restrooms for turning on water faucets, dispensing paper towels, etc., or sensors on sanitizing liquid dispensers) may be present on the device for detecting an input from a user, such as by waving their hand in front of the sensor. The sensor may be located anywhere on the device that is accessible to the user but may be preferably located on a front outer surface of the device facing the user.

Alternatively, however, no sensor may be used with some embodiments of an apparatus having a mechanism for holding and releasing a writing implement(s). Instead, a timer may operate to expose the writing implement(s) to disinfecting energy or light for a predetermined, sufficient and/or minimum amount of time, and the writing implement may then be released by the holder, such as to fall to a tray, after sufficient disinfecting or sanitizing has occurred. Such a timer may be part of a controller of a device. Implied in having a timed or user-driven release of a writing implement(s) is a mechanism for holding the writing implement(s) inside the chamber of the apparatus. However, it is conceivable that sufficient disinfection may be achieved by a writing implement(s) passing through the interior irradiated chamber only transiently, such as by gravity. Time of travel through the interior of the apparatus may be increased by slower rolling of the implement through the device, such as down an inclined surface(s) at various angles, rather than at faster or near free fall speeds.

When an input is detected or received by a sensor (or via a mechanical input), an actuator in response to such an input may cause movement of a holder from a first position (engaged with or contacting the writing implement) to a second position (to disengage or release the writing implement) and thus allow the writing implement to exit the interior chamber of the device, such as by gravity, so that a user may access and pick up the implement for use, such as from a receiving tray. The actuator may be any device capable of causing movement of the holder, such as an electric motor, servomotor, etc. Depending on the holding mechanism, after the implement(s) is/are released, the holder may then return to its original first position to contact, engage and/or retain another writing implement(s) inside the chamber until the next input is received from a user. According to these embodiments, the holder may function in a cycle between a first position and a second position to release writing implements successively one at a time, and the speed of movement of the holder between the first and second positions during the cycle may help to selectively release only a predetermined number of writing implement(s) (e.g., one implement) in response to an input, perhaps while retaining other writing implements that may also be present inside the chamber. According to some embodiments of the present invention, detection of a “request” by a sensor or other input may not result in release of a writing implement until a predetermined amount of time has passed since the writing implement was inserted into the device (especially in cases where a single writing implement is reused) to ensure that sufficient disinfecting has occurred before its release. For example, a sufficient level of UV irradiation around a writing implement may be able to kill more than 90% of microbes on the implement after about 15 to 20 seconds.

The holder may have any suitable size and shape depending on the precise mechanism and its location within the device, and it may operate according to any suitable mechanism to switch or advance between retaining and releasing states for a writing implement in response to an input. In fact, the holder may comprise two or more holders that may work in combination (e.g., positioned at different positions along or near the longitudinal axis of the implement. A holder may operate in a reversible or non-reversible cycle. For example, the holder may be an arm, bar, lever, extension, projection, etc., that may be rotated, extended/retracted, etc., from a first (engaged or retaining) position to a second (disengaged or releasing) position to release a writing implement. Subsequently, the holder may revert back again to the first position to retain or hold the next writing implement.

According to other embodiments, the holder may be a disk, sprocket or wheel, which may be oriented vertically, that does not cycle reversibly between two states (i.e., holding and releasing). Instead, the holder may advance or cycle through a series of two or more states or positions non-reversibly, which may eventually be repeated. But, unlike a reversible two-state holder, advancing the holder from a first state to a second state may release a first writing implement but also serve to hold another (second) writing implement in the second state. As an example, a vertically oriented disk, sprocket or wheel-like holder may have indentations, notches or the like to engage an individual writing implement and advance its movement and/or cause its release by rotation. Such a non-reversible holder may have only two states or positions, but the path or direction of movement of the holder from the second position to the first position would be different than the path or direction of movement from the first position to the second position (unlike a reversible cycle between two positions). Such a wheel-like or sprocket holder may be located anywhere within the chamber from the bottom opening upward but will preferably be located high enough such that the writing implement(s) held inside the chamber will be irradiated by the high energy waves or light. The holder may be connected or associated with one or more of the front portion, the back portion and/or the side portion(s) of a device. For example, if the holder rotates between first and second (or more) positions, then a pivot of the holder may be on or near such connected or associated portion(s) of the device.

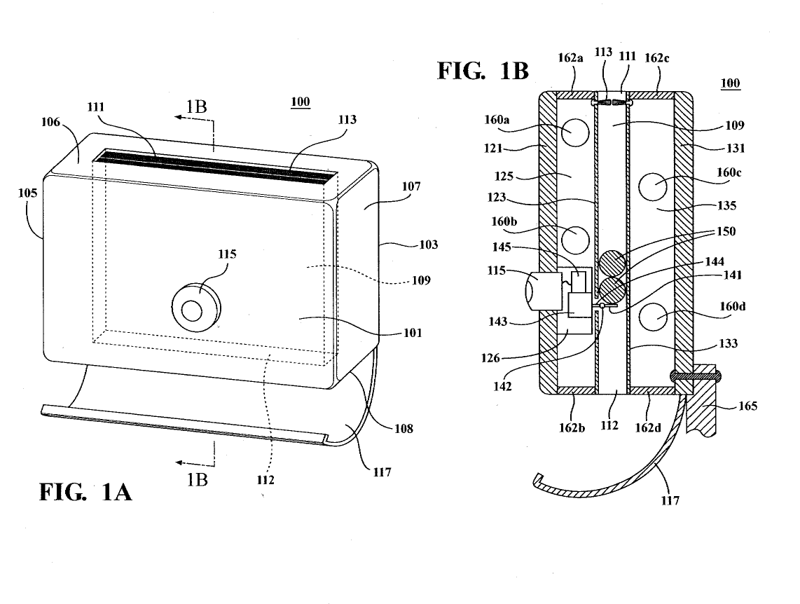

FIG. 1 shows a device 100 of the present invention according to some embodiments. The device 100 in FIG. 1A is shown comprising a front portion 101, a back portion 103, a left side portion 105 and a right side portion 107. The front, back and side portions 101, 103, 105, 107 of the device 100 are also shown enclosed at the top 106 and bottom 108 of the device 100. Enclosed within these portions is an interior chamber or enclosure 109 for receiving the writing implement(s) 150, inserted through a top opening 111 at the top of the chamber 109 (shown outlined in dotted lines). The writing implement(s) inserted into the top opening 111 may travel through the chamber 109 and exit the device 100 through a bottom opening 112 of the chamber 109. The enclosed top 106 of the device 100 is shown surrounding the top opening 111, and the enclosed bottom 108 of the device 100 is shown surrounding a bottom opening 112. A motion sensor 115 is also shown on the front surface of the front portion 101 of the device 100 to detect an input from a user. A tray 117 is further shown underneath the device 100 for receiving and catching a writing implement exiting or ejected from the device 100 in response to a user input.

A shield 113, which may be formed of a brush, flap, compressible material, etc., may be present at or near the top of the chamber 109 (e.g., at or near the top opening 111) for blocking the high energy rays or light from the interior of the device 100 that might be harmful or unsafe for a user or others in the vicinity. The shield 113 may be bendable or retractable to allow a writing implement to be deposited or pushed through it and into the chamber 109, but also resilient enough, such that the shield 113 then reverts or returns back to its light-blocking position after the writing implement(s) is pushed through it. As shown in FIGS. 1A and 1B for example, the shield 113 may comprise two rows of opposing brushes. Another shield may also be present near the bottom of the chamber 109 (e.g., at or near the bottom opening 112) if it does not interfere with the writing implement(s) 150 falling or ejecting out of the device 100 through a bottom opening 112 of the chamber 109. For example, a bottom shield may include a diagonal deflector that is angled rearwardly downward from a front side of the bottom opening 112 of a device to block harmful light or rays traveling forward toward a user in front of the device without interfering with the implements exiting the device. The lower part of the chamber 109 near the bottom opening 112 may alternatively or additionally be turned or curved away from a user to block or minimize direct paths of light from the device through the bottom opening 112.

FIG. 1B shows a cross-sectional view through the device 100 in FIG. 1A from a plane depicted as line and arrow 1B. Front portion 101 may have an outer wall 121 and an inner wall 123 with a front compartment 125 between them. Likewise, back portion 103 may have an outer wall 131 and an inner wall 133 with a back compartment 135 between them. The space shown between the front inner wall 121 and the back inner wall 131 is the interior chamber 109. For purposes of the present invention, the term “outer wall” is not limited to a single wall or partition but may include one or more walls or partitions, perhaps in addition to other structures or components. The shield 113 is shown near the top of the chamber 109 and the top opening 111 of the chamber 109 to help block harmful radiation from exiting the top opening 111.

Writing implements 150 are shown held inside chamber 109 by a lever or arm-like holder 141. The holder 141 may be moved (or rotated about a pivot 142; e.g., downward, etc.) by an actuator 143 from a first position (as shown) to a second position that is out of the way to allow a writing implement to fall down by gravity into a tray 117 for access by a user. The holder 141 may then return to its original first position to hold any remaining writing implement(s) in the chamber 109. For example, operation of the actuator or motor 143 may cause rotation of the pivot 142 via a drive loop 144, such as a small drive chain, belt, etc., in either direction to cause turning or angular movement of the lever or arm-like holder 141 between the first holding position and the second releasing position (and back again). The pivot 142 may be a rod, pin, shaft, etc., mounted to the device 100, to which the holder 141 is fixedly attached. Operation of the actuator 143 may be controlled by a controller 145. The controller 145 may be a computer, microprocessor, microcontroller, circuitry, etc., that controls the operation of the actuator 143 to control the timing and movement of the holder 141. The controller 145 may also receive a signal or input from the sensor 115, such as by a wired connection, and possibly interpret this input to determine when to cause movement of the holder 141. The controller 145 may also control the high energy sources or lamps 160, such as via a wired connection, to determine when they are turned on/off as well as the timing or pattern of irradiation (e.g., pulsed, continuous, periodic, etc.) including the length of time, and/or possibly the intensity level or wavelength(s) of the light or energy from the sources or bulbs.

The internal portion of the sensor 115, the controller 145 and the actuator 143 may be contained in an additional enclosure(s), which may be within or spanning the outer wall 121 and/or the front compartment 125 of the front portion 101. For example, these components are shown in an additional enclosure 126 in FIG. 1B. However, it is important to note that the components of the holding mechanism including the sensor 115, controller 145, holder 141, actuator 143, etc., may be positioned at different locations within the device 100, and each of these components of the holding mechanism may themselves be located in different places within the device 100 that are separate and apart from each other. The control components of the device may also be connected via a wireless communication. Indeed, the components of the holding and controlling mechanism are shown in simple or block form to indicate that the precise mechanism and the locations and types of components may vary. Although FIG. 1B shows the components of the mechanism in the front portion 101, any one or more of these components may instead be located in the back portion 103 and/or a side portion 105, 107, although the sensor 115 may preferably be located in the front portion 101 of the device 100 for greater accessibility. Although not shown, a device of the present invention may also have one or more internal sensors or switches, such as for detecting the insertion and/or presence of a writing implement(s) inside the chamber of the device, such as to initiate irradiation of the implement(s). A writing implement may trigger a mechanical or optical sensor upon entrance into, or passage through, a portion of the chamber, which may be communicated to a controller of the device for triggering irradiation of the implement(s). For example, the shield at or near the top opening of a chamber may also function as a sensor to trigger turning on of the high energy or UV source(s) or lamp(s), such as for a predetermined period of time.

Generally, multiple high energy sources or bulbs may be present in the front and back compartments 125, 135 of the device 100, such that all (or nearly all) of the peripheral surfaces of the writing implement(s) may be more effectively irradiated by the high energy light from many different angles. Four high energy sources or bulbs 160a, 160b, 160c, 160d, such as UV bulbs, are also shown in FIG. 1B in the front portion 101 and back portion 103 of the device 100 (two bulbs for each of the front and back portions). These sources 160 are used to irradiate the writing implement(s) 150 inside the chamber 109 with high energy light from them that passes through the transparent front and back inner walls 123, 133. Although not shown in FIG. 1, sockets, such as pin sockets, etc., may be present on the side(s) of the front and back compartments for attaching and plugging in the high energy or UV bulbs in a horizontal orientation, and/or those bulb sockets may be present on inwardly extending platforms within those compartments. With other embodiments having vertically arranged bulb(s), the sockets may be present on the top and/or bottom of the front, back and/or side compartments.

As mentioned previously, the number and arrangement of these energy sources or bulbs 160 may vary. These sources or bulbs may be located in one or more of the front, back and/or side portions of a device, and there may be one or more such sources or bulbs in each portion, if any, with each of the sources or bulbs arranged or positioned in potentially any suitable orientation relative to the dimensions of the device. According to embodiments of the present invention, bulbs in the different portions of a device may be arranged or positioned in any pattern, such that that they are staggered, offset, aligned, parallel, diagonal and/or perpendicular in relation to each other. As further shown for example in FIG. 1B, top and bottom caps 162a, 162b, 162c, 162d may be present near the top and bottom of each of the compartments 125, 135 to enclose them, which may also form part of the top 106 and the bottom 108 of the device 100. A reflective material may also be present on the inner surface(s) of one or more of these top and bottom caps for redirecting and concentrating the high energy light toward the writing implement(s) inside the inner chamber 109.

Although not shown in FIG. 1, devices of the present invention will also generally have a power source to power the energy-emitting sources or light bulbs and other electronic and mechanical components of the device, such as the sensor, controller and actuator. The power source may include a power cord that may plug into a wall outlet and/or one or more batteries, which may include rechargeable or disposable batteries. The power source may also be another electronic device including an electronic device attached to, and/or associated with, the device of the present invention, such as a credit card machine or an electronic signature or data entry pad. In addition, a support 165 is shown in FIG. 1B, which may include a stand, such as for setting the device on a table top, counter or other flat or inclined surface, or any other structure for attachment or coupling to another device, stand, etc. As another example discussed further below, the support 165 may attach to another electronic device, such as a credit card swiping machine or an electronic signature or data entry pad including, for example, those manufactured by VERIFONE® or HONEYWELL®, for their use together. The support 165 is shown in FIG. 1B attached to a lower part of the back portion of the device. However, a support structure according to other embodiments of the present invention may be attached elsewhere to the device and/or may be integral with, or continuously part of, one of the portions of the device.

FIG. 1C shows a cross-sectional view of another embodiment of a device 200 of the present invention that is similar to the device 100 in FIG. 1B but having a different mechanism for holding and releasing the writing implement(s) 250 inside the inner chamber 209 of the device 200. The front portion of the device 200 is again shown having an outer wall 221 and an inner wall 223 with a front compartment 225 between them. Likewise, the back portion of the device 200 is shown having an outer wall 231 and an inner wall 233 with a back compartment 235 between them. The space shown between the front inner wall 221 and the back inner wall 231 where the writing implement(s) 250 may be placed, held, etc., is the interior chamber 209. Again, the term “outer wall” is not limited to a single wall or partition but may include one or more walls or partitions, perhaps in addition to other structures or components. A shield 213 is shown near the top of the chamber 209 and the top opening 211 of the chamber 109.

According to this embodiment, writing implements 250 are shown held inside the chamber 209 of the device 200 by a wheel-like or sprocket holder 241 having a plurality of equally-spaced apart radial arms for engaging and holding the writing implement(s) while the holder 241 is stationary. The outer portions of the arms of the sprocket holder 241 may be moved (i.e., rotated about a pivot 242) by an actuator 243 a predetermined angular distance to allow a writing implement 250 to fall down by gravity into a tray 217 for access by a user without releasing any additional writing implement(s) from within the device 200. Thus, the actuator or motor 243 may cause incremental movement or rotation of the holder 241, such as via a drive wheel 244a and coupling arm 244b with the coupling arm 244b being pivotally mounted off-center from the axes of rotation of the drive wheel 244a and sprocket holder 241. By rotation of the sprocket holder 241, the outer portions or the arms of the sprocket holder 241 may move a sufficient angular distance to release a writing implement(s). The pivot 242 may be a rod, pin, shaft, etc., mounted to the device 200, to which the wheel or sprocket holder 241 is attached. As an alternative mechanism, the actuator or motor may cause incremental movement or rotation of the pivot via a drive loop (e.g., a small drive chain, belt, etc.), which may be coupled or engaged with the pivot, to cause corresponding rotation of the sprocket holder.

As indicated in FIG. 1C, a wheel-like or sprocket holder may have multiple radial arms that are angularly spaced-apart and project outward from a central portion of the holder, the central portion fixedly attaching to a pivot, such as a rod, pin, shaft, etc. For example, the sprocket holder 241 in FIG. 1C is shown having four angularly spaced-apart radial arms for engaging the writing implement(s). Thus, in a first instance for example, an outer portion of at least a first radial arm of the sprocket holder 241 may contact and hold a first writing implement 250a inside the chamber 209 of the device 200. Upon limited movement of the holder 241 by the actuator 243 via the drive wheel 244a and coupling arm 244b, the sprocket holder 241 may be rotated such that the radial arms of the holder 241 are moved an angular distance to release the first writing implement 250a. However, such rotation of the holder 241 may also move a second radial arm of the holder 241 into the approximate position previously occupied by the first radial arm to possibly catch and hold a second writing implement 250b, which may have been previously stacked above the first writing implement 250a. Although only one sprocket holder 241 is shown in FIG. 1C, multiple sprocket holders may be present within a device of the present invention, such as at different points along the length of a writing implement.

The amount of angular distance traveled by the radial arms of a wheel-like or sprocket holder during each of its incremental turns or rotations (i.e., to release one writing implement at a time) may depend on how many radial arms are present on the holder and whether they are equally spaced-apart. However, for a holder having equally spaced-apart radial arms, the amount of incremental movement may be approximately equal to 360°/n, with n=number of radial arms. Thus, the sprocket holder 241 in FIG. 1C with four radial arms may be rotated approximately 90° with each incremental turn to release a writing implement.

Operation of the actuator 243 of the device 200 in FIG. 1C may be controlled by a controller 245. The controller 245 may be a computer, microprocessor, microcontroller, circuitry, etc., that controls the operation of the actuator to control the timing and movement of the pivot 242 and sprocket holder 241, such as via a drive loop or the coupling arm 244b as shown. The controller 245 may also receive a signal or input from a sensor 215, such as via a wired connection, and interpret this input for causing movement of the holder 241. The controller 245 may also control or determine when the high energy sources or lamps 260 are turned on/off as well as the timing or pattern of irradiation (e.g., pulsed, continuous, periodic, etc.) including the length of time, and/or possibly the intensity level or wavelength(s) of the light or energy from the sources or bulbs. To accommodate the sprocket holder 241 of the device 200, a cavity 210 may be present and continuous with a front part of the chamber 209, which may be bounded by a cavity wall 224 that may be continuous with the inner wall 223 of the front portion and front compartment 225 of the device 200. A gap or slot may be present in such a cavity wall 224 to allow a drive loop or coupling arm 244b to reach through and attach or couple to the pivot 242 and/or holder 241. Depending on the arrangement, there may be multiple chamber cavities and/or multiple gaps or slots through the cavity walls of one or more chamber cavities along a side-to-side axis of the chamber of a device for multiple sprocket holders. Alternatively, two or more sprocket holders may be inside the same cavity of a device chamber. Again, a cavity, sprocket holder, actuator, coupling arm or drive loop, etc., may alternatively be present on a back side of the chamber of a device and/or continuous with a back inner wall.

The internal portion of the sensor 215, the controller 245 and the actuator 243 may again be contained in an additional enclosure(s), which may be within or spanning the outer wall 221 and/or the front compartment 225 of the front portion of the device 200. However, it is important to note that the components of the holding mechanism including the sensor 215, controller 245, holder 241, actuator 243, etc., may be positioned at different locations within the device 200, and each of these components of the holding mechanism may themselves be located in different places within the device 200 that are separate and apart from each other. Again, the components of the holding mechanism are shown in simple or block form to indicate that the precise mechanism and the locations and types of components may vary. Although FIG. 1C shows the components of the holding and releasing mechanism in the front portion of the device 200, any one or more of these components may instead be located in the back portion and/or a side portion(s) of the device 200, although the sensor 215 itself may preferably be located in the front portion of the device 200 for greater accessibility by a user.

Multiple high energy sources or bulbs may be present in the front and back compartments 225, 235 of the device 200 for more effective irradiation or most or all of the surfaces of the writing implement(s). Four high energy sources or bulbs 260a-d, such as UV bulbs, are shown in the embodiment in FIG. 1C in the front and back portions of the device 200 (i.e., two bulbs in each of the front and back compartments 225, 235). High energy light generated by these sources or lamps 260 may irradiate writing implement(s) 250 inside the chamber 209 by passing through the transparent front and back inner walls 223, 233 of the device. As mentioned previously, the number and arrangement of these sources or bulbs may vary. These energy sources or bulbs may be located in one or more of the front, back and/or side portions of the device 200, and there may be one or more such sources or bulbs in each of those portions or compartments of a device, if any, with each of the energy sources/bulbs arranged or positioned in potentially any suitable orientation relative to the dimensions of the device.

According to embodiments of the present invention, bulbs in different portions of a device may be arranged or positioned in any pattern, such that that they are staggered, offset, aligned, parallel, diagonal and/or perpendicular in relation to each other. The high energy sources or bulbs may be arranged vertically, horizontally or diagonally in one or more of the compartments of a device. For example, high energy or UV bulbs may be arranged vertically in a front compartment and horizontally in a back compartment, or vice versa, of a device.

Devices of the present invention may also generally be configured to block or minimize the amount of high energy or UV light exiting the device. Although embodiments of the present invention may generally include one or more high energy sources or bulbs for producing the high energy radiation to disinfect, etc., the writing implement(s) inside the chamber of a device, such high energy radiation may also or instead be produced by an exterior device (or remote source) and optically transmitted to the interior chamber of the device, such as through fiber optics, mirrors, lenses, etc. Although not shown in FIGS. 1B and 1C, a device may also have a portion of the top and/or bottom opening(s) of the device partially enclosed (apart from the shield) by an additional wall, cap, structure, etc.

The embodiments shown in FIG. 1 are simplified examples to show some of the basic features of the present invention. It is important to note, however, that many of these features may be different and/or combined with other features not shown. According to many embodiments, the present invention may generally include any enclosure that can accommodate writing implement(s) to expose those writing implement(s) to high energy or light, such as UV light, emitted and/or originating from within the enclosure to have an antimicrobial or antibacterial effect on the surfaces of the writing implement(s). Devices of the present invention may also generally be configured to eject a writing implement in response to a user input. Thus, it is contemplated that devices of the present invention may have any of a variety of possible configurations, shapes, sizes and component combinations that would achieve these general functions and purposes. For example, even though the devices in FIG. 1 are shown having generally rectangular dimensions, devices of the present invention may have other shaped dimensions including non-linear, non-symmetrical and/or non-geometric shapes along with other design elements without departing from the spirit and scope of the present invention, and the sides, outer walls and/or inner walls of various devices of the present invention may have any of a variety of sizes and shapes and may not be parallel to each other. A device of the present invention may also have a front portion and a back portion, but without one or both of the side portions, such as where the front and/or back portions of the device are curved or non-planar and/or meet by convergence or at a point.

The interior or inner chamber of a device must generally be configured, sized and shaped to receive and accommodate the shape of one or more writing implements inserted into the device and allow those writing implement(s) to travel through the device by gravity (in the absence of a mechanism for actively moving or conveying the implements). Thus, the cross-sectional size and shape of the interior chamber may depend on the dimensions of the writing implement(s) to be placed inside the chamber. However, the inner chamber may be linear, sloped, curved, sigmoidal, sinusoidal, zig-zag, sawtooth, etc., in shape along the path of the inner chamber between the top and bottom openings of the device in at least one plane or dimension. It is also conceivable for a device to have a chamber that twists between the top and bottom openings such that the orientation of the width of the chamber turns or changes from top to bottom. While the shape of the path of the chamber of a device between the top and bottom openings may vary, the cross-sectional size and shape of the chamber (i.e., within a horizontal plane) may generally stay about the same along its top-to-bottom path, although some variation in size and shape is possible.

Raised bumps may be optionally present on one or more inner surface(s) of the chamber of a device (e.g., on the surface(s) of one or more of the inner wall(s) of the chamber) to encourage rolling or rotation of the writing implement as it travels down the path of the chamber. The raised bumps may be made of a rubber-like material that may “grip” the writing implement(s) to help cause their rotation or rolling. Rolling of the writing implement(s) may be especially useful for exposing all or most of the surfaces of the writing implement(s) when the high energy light sources or bulbs are present on only one side of the device.

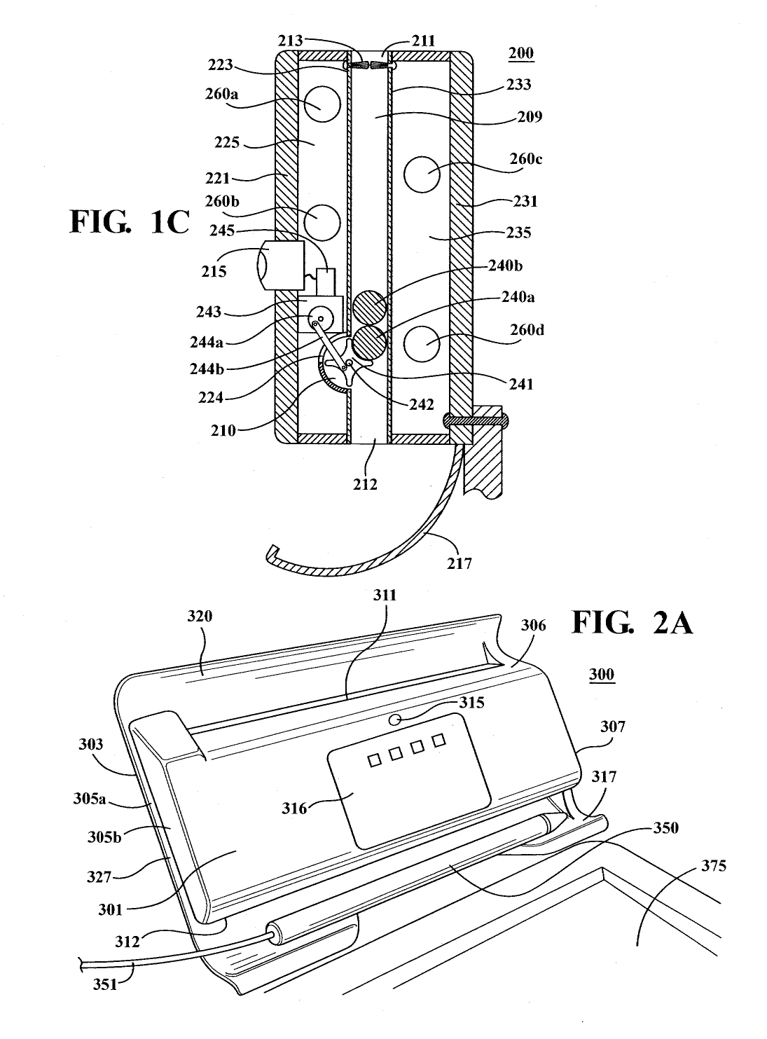

FIGS. 2A and 2B provide a perspective and side view, respectively, of another device embodiment 300 of the present invention for disinfecting, etc., a writing implement or stylus 350, which may be used with a standard electronic data entry, credit card machine and/or signature pad. A front portion 301, a back portion 303, a left side portion 305 and a right side portion 307 surround and at least mostly enclose an interior chamber for receiving the writing implement or stylus 350, which may be tethered by a cord 351 to an associated electronic device 375. The stylus 350 may be held in place within the chamber 309 by a holder mechanism (not shown) that may be rotated or moved out of the way to release the stylus 350 (see, e.g., FIGS. 1B and 1C described above), such that the stylus 350 falls down into the tray 317. The top opening 311 of the chamber 309 may have a shield to block or minimize any harmful light from exiting the top of the device 300. A backstop 320 may extend upwardly from the top of the back portion 303 of the device 300 to help catch and direct the writing implement or stylus 350 toward and into the top opening 311 of the inner chamber. Although not shown in FIGS. 2A and 2B, many of the interior features of the device 300 may be the same or similar to the device feature(s) described above in reference to FIGS. 1A-1C and/or as otherwise described herein.

The stylus 350 may have any suitable cord 351, such as a string, ball chain, etc., for tethering the stylus 350 to (i) the disinfecting device 300 itself, (ii) an associated electronic device 375, or (iii) another nearby surface. To allow the stylus 350 to enter into, and travel through, the chamber 309 of the device 300 without the tethering cord 351 getting stuck, tangled or in the way, a side slot 327 may be present in the side of the chamber 309 and connecting and spanning between the top and bottom openings 311, 312 of the chamber. Thus, the tethering cord 351 may travel with, and next to, the path of the stylus 350 through the side slot 327 of the device 300 with the stylus passing through the chamber of the device 300 (i.e., from the top opening 311 to the bottom opening 312). The side slot 327 may also have a shield(s), such as a pad(s), brush(es) or flap(s), which may be similar to the shield near the top opening 311 of the chamber to block or minimize harmful light exiting through the side slot 327. Due to the presence of the of the side slot 327, the left side portion 305 may in fact comprise a back left side portion 305a and a front left side portion 305b separated by the side slot 327. However, one or both the front and/or back left side portion(s) may be absent if a side slot is present between the back portion and the front left side portion, between the front portion and the back left side portion, or between the front and back portions of the device. A side slot may also be present on a right side portion of the device (instead of the left side portion), and the right side portion may be divided into front and/or back right side portion(s).

According to some embodiments, one or two opposing pad(s), which may be made of foam, silicone or similarly compressible material, may be present between the front and back inner surfaces of a side slot that may be compressed due to physical pressure or force, such as by passage of the tethering cord, but revert to its original more expanded shape (i.e., to close any spacing) when the force or pressure is removed. According to the embodiment 300 (as shown more clearly in FIG. 2B), two compressible pads 374, 376 may extend inwardly from the front and back inner surfaces of the side slot 327 and have opposing surfaces that are in contact (or nearly in contact), such that harmful light rays may be mostly or fully blocked from exiting through the side slot 327 of the device 300. However, as the tethering cord 351 travels between the top and bottom openings 311, 312 of the device (along with the stylus 350), the contacting surfaces of the two light-blocking pads 374, 376 are forced apart slightly by the thickness of the tethering cord 351 as it passes between them, but closure between the two pads 374, 376 is restored after the tethering cord 351 passes due to the pads 374, 376 being biased to maintain their original shape. Although the embodiment 300 in FIG. 2 shows two opposing pads 374, 376, only one pad may be used according to other embodiments that may contact a front or back inner surface of the side slot 375. One or two opposing flap(s), brush(es) or the like may also be used. However a stylus for an electronic data entry or touch pad may not have a tethering cord. In such cases, the side slot may be absent.

According to the device embodiment 300 in FIG. 2, one or more high energy sources or lamps, such as UV lamps, may also be provided within a front and/or back compartment for irradiating a writing implement or stylus 350 inside the chamber of the device 300. As with any of the embodiments, one or more additional replacement bulb(s) may be stored within an interior space, compartment, etc., of the device (not shown). An optional panel(s) or hatch(es) may be present on one or more side(s) (e.g., a back side) of the device that can be opened or removed, such as to access any replacement bulb(s) or power source and/or to change a source/bulb that has expired or burned out. Again, reflective surfaces may be used to trap and reflect the disinfecting light inside the device 300. A motion sensor 315 may be present on the outer face of the front portion 301 of the device 300 for detecting an input from a user. When an input is detected, an internal holder of the device 300 may be moved, rotated, etc. (depending on the exact holding mechanism), to release the stylus 350. Again, a power cord and/or one or more batteries may be used for powering the sources/lamps, sensor 315, internal holding mechanism, actuator, etc., of the device 300, such as by plugging into a wall outlet. The device 300 may also derive its power from an associated device, such as through a particular type of electric cord or other wiring. A support, bracket or mount 365 is also shown in FIG. 2B for supporting and/or attaching the device 300 to an associated electronic device. For example, a lower portion of the support 365 may be inserted into a receiving slot 377 of an associated device 375. A power on/off button, indicator lights and other decorative or design features 316 may also be optionally provided and displayed, such as on a front face of the first portion 301 of the device 300.

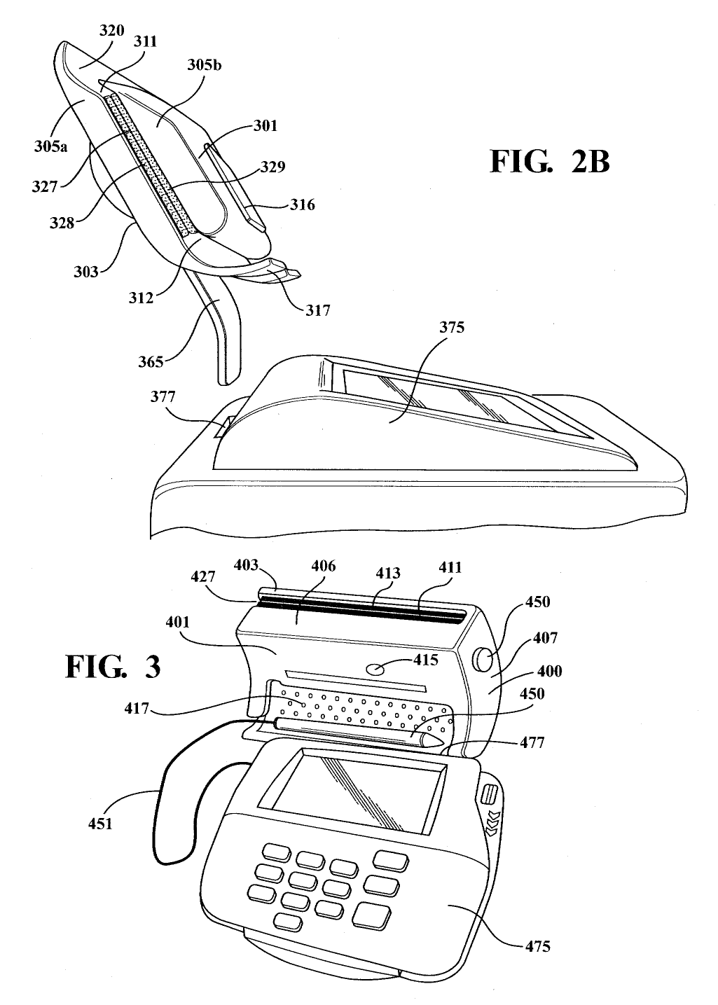

Another device embodiment 400 is shown in FIG. 3 that is similar in many ways to the device embodiment 300 in FIG. 2. The device 400 is shown having a front portion 401, a back portion 403, and a right side portion 407. However, a left side portion is absent due to a side slot 427 being present on the left side of the device between the front and back portions and spanning between a top opening 411 and a bottom opening of the chamber of the device 400. A shield 413 is further shown near the top opening 411 of the device 400 for blocking harmful radiation from the interior of the device 400. A tray or basin 417 is shown that is continuous with, and/or part of, at least the back portion 403 of the device 400. An optional motion sensor 415 is shown on the front face of the front portion 401 of the device 400 for release of a writing implement or stylus 450. The device 400 is shown attached by a hinged attachment 477 to an associated device 475, which may be a credit card swipe machine, etc., and the stylus 450 is shown tethered to the associated device 475 by a tethering cord 451. For the stylus 450 to pass through an interior chamber of the device 400 following its insertion through the top opening 411 of the chamber, a side slot 427 is provided for the tethering cord 451 to pass through it along with the stylus 450 passing through the adjacent chamber of the device 400. An optional power button 450 may also be present for turning the device 400 on/off.

The dimensions of a device of the present invention may vary. According to many embodiments, however, the width of a device between the outer walls of the left and right side portions of the device may be in a range from about 6 to about 10 inches, such as about 8 inches. The height of a device may be in a range from about 3 to about 6 inches, or about 4 inches, and the depth of a device may be in a range from about 1 inch to about 5 inches, such as about 3 inches. Within a horizontal cross-sectional plane, the inner chamber of a device of the present invention may generally have an elongated width (i.e., side-to-side dimension) corresponding to the elongated shape (i.e., length) of the writing implement(s) placed or deposited therein, and the depth (i.e., front-to-back dimension) of the chamber may generally be much less than the width of the chamber. Both the width and depth dimensions of the chamber of a device may be relatively constant between the top and bottom openings, although some variation is possible. Indeed, the horizontal cross-sectional size and shape of the inner chamber may be generally constant between the top and bottom openings, although again some variation is possible. According to many embodiments, the inner chamber may be about 5 to about 8 inches in width and have a depth from about ¼ inch to about 1 inch. According to some embodiments, the width of the inner chamber may be at least four times greater than the depth of the chamber as measured within the same horizontal cross-sectional plane, or alternatively the width of the inner chamber may be at least five times greater than the depth of the chamber, or alternatively the width of the inner chamber may be at least six, at least seven, at least eight, at least nine, or at least ten times greater than the depth of the chamber. The height or path length of the inner chamber between the top and bottom openings may also vary depending on a number of factors including the number of writing implement(s) that may be stacked inside the chamber, the type of holding and releasing mechanism, the amount of disinfecting radiation, the manner of movement of the writing implement(s) through the device, etc.

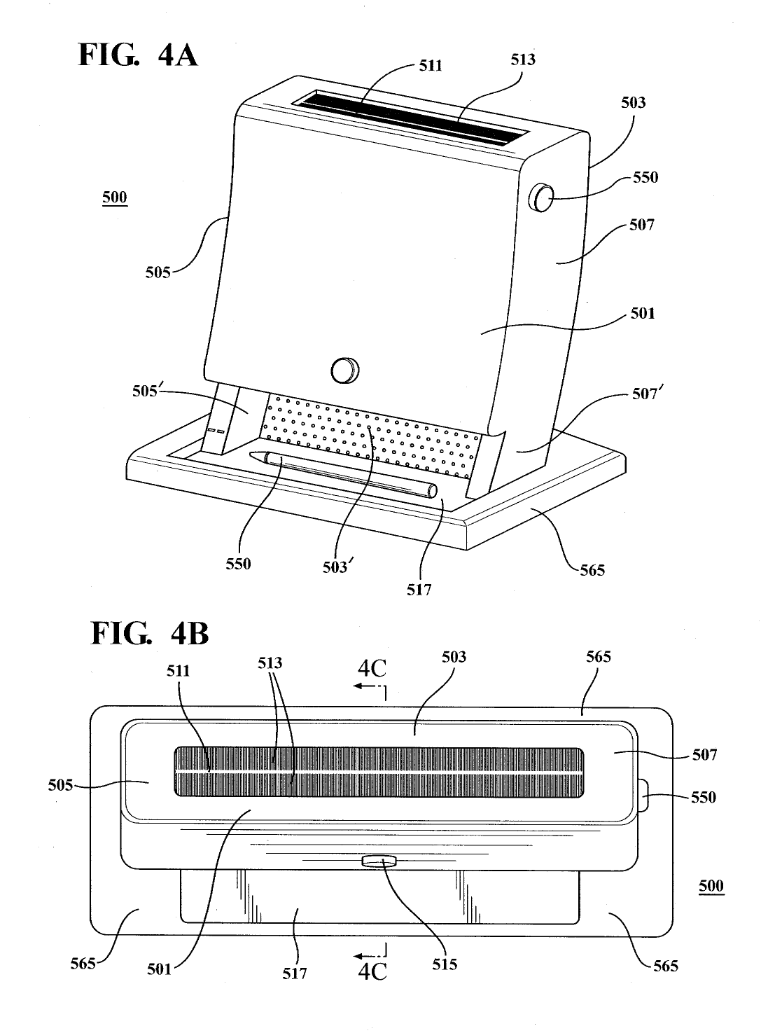

FIGS. 4A-4C show a set of views of another example device embodiment 500 of the present invention for holding a plurality of writing implements or pens 550 that may be stacked on top of each other inside the chamber 509 of the device 500. FIG. 4A provides a perspective view of the device 500, FIG. 4B provides a top view of the device 500, and FIG. 4C provides a cross-sectional view through the device 500 along a plane (labeled as 4C in FIG. 4B). The chamber 509 is enclosed by a front portion 501 and a back portion 503 in addition to left and right side portions 505, 507. A brush-like shield 513 is also shown near a top opening 511 of the chamber 509 for blocking harmful light from exiting the device 500. The top opening 511 may be tapered outward toward the top 506 of the device 500 to facilitate the deposit of writing implements into the chamber 509. Multiple UV lamps 560a, 560b, 560c, 560d are shown in the front portion 501 of the device 500 for irradiating the writing implements 550 inside the chamber 509. Similarly to above, reflective material or surfaces may be used inside the device 500 to direct or reflect the sanitizing or disinfecting light toward the chamber 509. Reflective material may be present in an inner surface of an outer wall of the front portion 501 of the device 500 and/or on the inner surface of the inner wall of the back portion 503 of the device 500. On the other hand, the inner wall of the front portion 501 of the device 500 may be transparent to allow the high energy light from the sources/lamps 560 to pass through and reach the writing implement(s) 550 inside the chamber 509 of the device 500.

A motion sensor 515 may also be present on the front portion 501 of the device 500 to receive an input from a user to release a writing implement 550. In response to receiving an input from a user, a writing implement 550 may be released or dropped from the device 500 such that is falls into the tray 517, which may be formed in the base of a support or stand 565. To control the release of the writing implement(s) 550 in response to an input, a holder 541 is shown in FIGS. 4A-4C that rotates (at least temporarily or momentarily) about a pivot 542, which may be on or near the back portion 503 of the device 500, from a first (retaining) position to a second (releasing) position. The holder 541 may then revert back to the first position to hold any remaining implement(s) 550 inside the chamber 509. An opposing projection or lip 540 may also be present, such as on or near the front portion 501 of the device 500, to work in conjunction with the holder 541, which may be on or near the back portion 503 of the device. In between the holder and the tray, a drop zone or path may be present that may be defined by lower extensions of the side, back and/or front portions of the device. For example, the device 500 in FIG. 4 has a drop zone or path with an open front side having a lower back extension 503′ of the back portion 503, a lower left side extension 505′ of the left side portion 505, and a lower right side extension 507′ of the right side portion 507.

A variety of different types of writing implement(s) may be used with the device in FIG. 4 depending at least in part on the dimensions of the inner chamber. Indeed, a device of the present invention may be able to separately or simultaneously accommodate writing implement(s) of different types and/or sizes. The dimensions of the device 500 in FIG. 4 or similar devices for holding multiple writing implements may vary. According to many of these embodiments, the width of a device between the outer walls of the left and right side portions may be in a range from about 6 inches to about 12 inches, such as about 7 to 9 inches or about 8 inches. The height of the device may be in a range from about 4 to about 12 inches, such as about 8 to 12 inches or about 10 inches. However, the height of the device may be shorter if fewer writing implement(s) are stored inside the device even if they may have a larger diameter. Basically, the height of the device (and inner chamber) may depend on the number implements to be stored at once and their combined diameters. The depth of the device may be in a range from about 1 inch to about 5 inches, such as about 2-3 inches or about 3-4 inches for larger writing implements. The inner chamber of the device may be sized to accommodate a particular writing implement(s) but may be deeper to accommodate larger (thicker) writing implements, such as highlighters or markers. A stand or support of the device may be about 1 to 4 inches or about 2-3 inches in height and about 2 to about 5 inches or about 3 to about 4 inches in depth.

According to another broad aspect of the present invention, a cradle-like device is provided having a receiving area or cavity for placing, receiving, etc., a writing implement thereon. Such a cradle-like device may have a receiving area or cavity that is enclosable by closing a cover or lid. However, the cradle-like device will generally lack an inner chamber with top and bottom openings as described above for other embodiments. The receiving area or cavity may be recessed and be open on top, such as when any cover or lid that might be present during irradiation of the implement(s) is removed or open. Indeed, a cover or lid may be opened or removed to allow a writing implement to be placed or set onto the receiving area or cavity may be opened or removed to allow the writing implement to be placed inside the device, but the cover or lid may be closed to contain the potentially harmful light or energy inside the device during irradiation of the writing implement. Generally, the opening for placing or removing the writing implement will be on the top of the receiving area so that the writing implement may be placed or laid inside the receiving area or cavity of the device and held in place by gravity.

A cradle-like device of the present invention may have a cradle portion and a housed portion. The cradle portion may have a receiving area or cavity, which may be recessed for holding the writing implement, a lower compartment that may house the high energy source(s) or lamp(s), which may be beneath the receiving area or cavity. The high energy light or waves from the source(s) or lamp(s) in the lower compartment may travel through a transparent window, which may be glass, plastic, etc., to reach and irradiate the writing implement in the receiving area or cavity above the lower compartment. Thus, the transparent window may separate the lower compartment from the upper receiving area or cavity of the cradle portion of the device. Thus, a cover or lid may be used to enclose the space around the writing implement above the receiving area or cavity of the cradle portion of the device. The cover or lid may optionally have reflective surfaces or materials to direct the high energy light or waves toward the other peripheral surfaces of the writing implement. The cross-sectional shape and dimensions of the receiving area may vary but may be sloped of curved to create a recessed area or cavity to hold the writing implement and keep it from rolling off. Such a recessed portion may be shallow or deep and may have any suitable cross-sectional shape including V-shaped, U-shaped, semicircular, trapezoidal, etc. Such a recessed area or cavity may also have upper and lower recessed area(s) of different or changing cross-sectional shapes and sizes.

The housed portion may be enclosed and contain a controller, a power source, such as batteries, and/or a power input, such as to receive power from a power cord plugged into an outlet. The housed portion of the device may contain a controller that may be in electrical communication with (i.e., in wired and/or wireless communication or connection with) one or more of a power source, the high energy source(s) or lamp(s), and/or a sensor(s). The sensor(s) may detect when the lid or cover of the device is closed (and/or open) for determining when the high energy source(s) or lamp(s), such as one or more UV lamp(s), may be turned on (i.e., when the lid or cover is closed) to avoid having a user or other person being exposed to harmful disinfecting radiation from the device. Thus, the controller may determine when the cover or lid is closed and/or the timing of when to turn on the high energy source(s) or lamp(s). Alternatively, the controller and its components may be integrated or incorporated into the cradle portion of the device. The housed portion and the cradle portion may be aligned end-to-end or arranged in a different way.

One or more high energy or light source(s) or lamp(s), such as UV lamp(s), may generally be provided within the interior of the device (e.g., in the lower compartment of the cradle portion of the device beneath the receiving area or cavity) and configured to irradiate the writing implement(s), such as through a transparent window. The cradle or receiving area or cavity of the device may be recessed for receiving and holding the writing implement. As mentioned above, the bottom of the receiving area or cavity of the cradle portion of the device may have a transparent window, such as glass, plastic, etc., to allow disinfecting or sanitizing light to pass from a lower compartment of the device (underneath the receiving area where the one or more high energy sources or lamps may be located or positioned). Alternatively or additionally, one or more high energy or light source(s) or lamp(s), such as UV lamp(s), may be located or positioned above the receiving area of the cradle portion of the device, such as on, or associated with, the top cover or lid. In such a case, a transparent window may not be necessary.

Regardless of the precise location(s) of the one or more high energy sources or lamps, the sources or lamps may be activated or turned on when the cover or lid is closed or shut to prevent or minimize harmful light or energy from exiting the device. The cover or lid may be detachable or hinged on one side to allow for its opening and closing, and the device may have a sensor, such as a depressible sensor, to detect when the cover or lid is closed to activate, enable, and/or turn on the source(s) or lamp(s).

According to some embodiments, such a cradle-like device of the present invention may have more than one (e.g., two or three) receiving areas for receiving more than one type of writing implement at the same time or sequentially. This may be useful in allowing alternating use of two different writing implements. Thus, a first writing implement may be used while a second writing implement is being cleaned or sanitized inside the device, and the second writing implement may then be removed later from the device and used while the first writing implement is placed inside the device to be cleaned or sanitized. Another potential use of such a device of the present invention having more than one receiving area is to accommodate different types of writing implements or sets of writing implements, such as a pen and mechanical pencil or different colored pens, pencils, markers, etc.

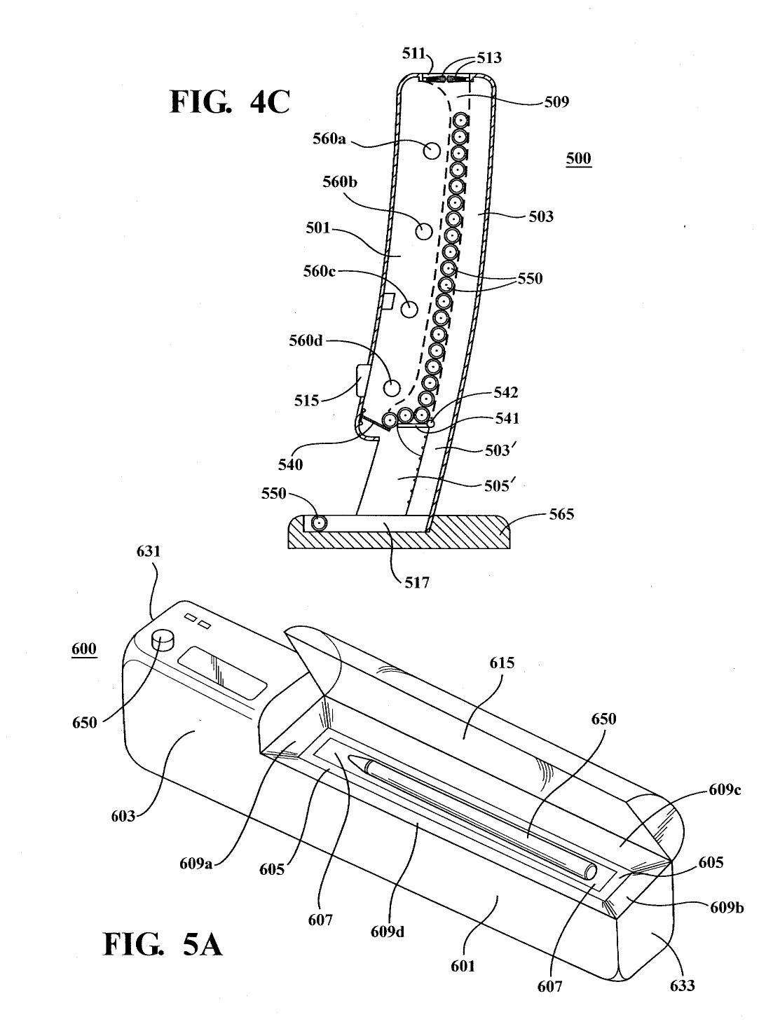

FIGS. 5A-5C show different views of an example embodiment of a cradle device 600 of the present invention, the device 600 having a cradle portion 601 and a housed portion 603, the cradle portion including a receiving area or cavity 605 with an open top for receiving and holding a writing implement 650 being placed thereon. FIG. 5A is a perspective view of the device 600 with a writing implement 650 placed thereon, FIG. 5B is a cross-sectional view of the device 600 through plane 5B in FIG. 5C, and FIG. 5C is a longitudinal cross-sectional view of the device 600. The receiving area 605 is recessed below and between the tops of the sides of the cradle portion 601 of the device 600 including the inwardly sloping walls 609a-d of the cradle portion 601. By having the receiving area 605 recessed below the sides and/or sloping walls 609a-d of the cradle portion 601, the writing implement 650 may be more securely held in place. A transparent window 607 is also shown as part of and below the recessed area 605 to allow disinfecting or sanitizing light from a high energy source or bulb 612 in a lower compartment 611 of the cradle portion 601 of the device 600 beneath the receiving area 605 to reach the writing implement 650. When a writing implement 650 is placed on the receiving area 605 of the device 600, a cover or lid 615 may be shut, closed or placed over the receiving portion 605 to form an enclosed space 606 around the writing implement 650 and above the receiving area 605. A high energy bulb 612 in the lower compartment 611 of the cradle portion 601 of the device 600 may be held in place by one or more bulb supports or stands 613, although the manner of attachment, support, socket, etc., of a bulb may vary.

The UV lamp or bulb 612 may only be turned on when the cover or lid 615 is shut or placed over the receiving area 605 to form the enclosed space 606 to prevent or minimize any harmful light from escaping the device 600. A controller 621, such as a computer, microprocessor, microcontroller, circuitry, etc., may also be present in the housed portion 603 of the device 600 to control when the UV lamp or bulb 612 is turned on/off, and/or to receive an input or signal from a lid/cover sensor(s) (not shown). To power the UV lamp or bulb 612 and any other electronic components, such as the controller 621, one or more batteries 623 may be provided in the housed portion 603 of the device 600, and/or a power cord may be provided that feeds into the device 600. Each of the electronic or powered components including the controller 621, power source, such as batteries 623 or power cord, and the bulb(s) 612 may all be connected by wires 625. Reflective surfaces may be provided throughout the interior of the enclosed space 606 of the device 600, such as on the inner surface(s) of the lid/cover 615 and/or the receiving area 605 (but not on the transparent window 607), to contain the UV light inside the device 600 and/or direct or reflect the UV light toward the writing implement 650. Other functional and design features may also be present.

The exact dimensions of a device according to FIG. 5 or similar devices for sanitizing or cleaning a writing implement(s) may vary. However, a cradle-like device having a linear arrangement of the cradle and housed portions, the total length of the device from one end of the housed portion to the opposite end of the cradle portion (e.g., from the first longitudinal end 631 to the second longitudinal end 633 of the device 600 in FIG. 5) may be in a range from about 7 inches to about 15 inches, such as about 8 to 11 inches. The height of the device may be in a range from about 1 to about 4 inches, such as about 2 to 3 inches. The depth of the device may be in a range from about 1 inch to about 5 inches, such as about 1-4 inches or about 2-4 inches. This depth may be greater if there is more than one receiving portion for more than one writing implement to be stored inside the device at the same time.

The length of the receiving area or cavity may generally be much greater than the depth of the receiving area or cavity. Indeed, the length of the cradle portion of the device may generally be greater than its depth to correspond to the dimensions of a writing implement. Both the length and depth dimensions of the receiving area or cavity may be relatively constant throughout its area, although some variations are possible. According to many embodiments, the length of the receiving area or cavity of a cradle-like device may be at least four times greater than its depth, or alternatively the length of the receiving area or cavity of a cradle-like device may be at least five times greater than the depth of the chamber, or alternatively the length of the length of the receiving area or cavity may be at least six, at least seven, at least eight, at least nine, or at least ten times greater than the receiving area or cavity.

When a writing implement is present in, on, inside, etc., a chamber or receiving area of any of the devices of the present invention, including those devices in FIGS. 1-5, the one or more high energy sources or lamps, such as one or more UV lamps, may be turned on and off continuously, periodically, intermittently, or in pulses, which may be controlled by a controller of the device as mentioned above. The intensity of the light or energy produced or emitted by the one or more high energy sources or lamps may also be varied over time. The period of time that the one or more high energy sources or lamps may be turned on may vary. According to some embodiments, for example, the one or more high energy sources or lamps may be activated and turned on only when or after a writing implement is inserted or deposited in or on a chamber, area or cavity of a device. According to some of these embodiments, the one or more high energy sources or lamps may then be activated or turned on only for a predetermined amount of time (e.g., a time sufficient to disinfect/sanitize) and then turned off. According to other embodiments, the one or more high energy sources or lamps may be activated or turned on after a writing implement is ejected following a user input. As mentioned above, high energy light may also be provided from an external source and guided into the device by optical equipment. In such a case, the timing and intensity of the irradiation may be achieved by controlling how and when such externally produced light is provided to the interior chamber.

Among the possible design and functional features for devices of the present invention, a protective glass or plastic “see-through” may be provided as part of any of these devices, including any of those shown in FIGS. 1-5, that blocks the UV or harmful light but allows the visible light from the high energy light sources or lamps to pass through it. This see-through window may have any of a variety of dimensions and shapes and be located anywhere on the device within sight of the user. This feature may provide not only aesthetic benefits but also visual confirmation that the light source(s) are “on” and working, which may be appealing to a user and indicate when a bulb needs to be replaced.

According to another broad aspect of the present invention, writing implements are provided that are specifically designed for use with disinfecting or sanitizing devices of the present invention. The length and diameter of a writing implement of the present invention may be suitably sized and shaped to fit the inside dimensions of the chamber of a corresponding device and travel through the chamber of the device without getting stuck. In traveling between the top opening and the bottom opening of the chamber, a writing implement may fall by gravity or roll along an inner wall(s) of the chamber. Such a writing implement may have a mostly or fully consistent diameter along its length or longitudinal dimension (i.e., mostly or fully cylindrical) and a length that is less than the width of the chamber. Features that extend or project outside the diameter of the writing implement, such as clips, caps, etc., may be absent to avoid the possibility that they might interfere with the writing implement traveling through the chamber and/or becoming disinfected, etc.

If a cap is used with the writing implement, the writing implement may have a smaller diameter and/or taper near its writing end or tip such that the cap may be flush with the rest of the writing implement (i.e., have about the same diameter as the rest of the writing implement). Instead of a cap to cover the writing tip, a push button at the end of the writing implement opposite the writing end or tip may be present that does not change the diameter or profile of the writing implement. Such a push button could be pressed repeatedly to extend and retract the writing tip. Such writing implements of the present invention may also have a smooth surface and/or be made of a UV-resistant plastic. Writing implement(s) of the present invention may also be made of material(s), such as plastics, etc., having antimicrobial properties, such as by having antimicrobial or antibacterial substances in, or on the surface of, such material.

According to some embodiments, writing implements of the present invention may have separating structures or separators, such as spaced-apart fins, flanges or rings, which may be used with a device(s) of the present invention, such as to separate adjacent writing implements inside the chamber of a device and prevent their touching with one another over a substantial surface area. This may help to minimize any cross-contamination between adjacently stacked implements inside the chamber of a device and/or allow the disinfecting light to reach more of the outer surfaces of the writing implements that might otherwise be “shaded” between the adjacently stacked writing implements inside the chamber of a device. Such fins, separators, etc., may include two or more fins, separators, rings, etc., with each fin, etc., with each ring being located closer toward a respective longitudinal end of the writing implement (i.e., one fin, etc., is closer to the writing end or tip, while another fin, etc., is closer to the opposite longitudinal end of the implement). Moreover, the spacing between adjacently stacked writing implements inside a device of the present invention may also be increased by such fins, separators, etc., to facilitate the individual release of a writing implement form the device by providing more of a gap or spacing between the stacked implements, which may provide more room for the holder of a device to catch the next writing implement above the one being released (i.e., by facilitated insertion of the holder into such a gap between the adjacently stacked wiring implements). Such gap, spacing or separation between adjacent implements may avoid having the holder of a device becoming jammed if the implements are too close together.

These fins, etc., may have a variety of different orientations, shapes, and sizes but may generally have only a slightly greater diameter than the rest of the outer surface(s) of the writing implement. As for their positioning, a first fin, etc., of a writing implement may be near, or closer to, a first longitudinal end of a writing implement, whereas a second fin, etc., may be near, or closer to, a second longitudinal end of the writing implement. By having sufficient spacing between at least two fins, etc., of an implement(s), wobbling of the implement(s) inside the chamber of a device may be avoided. The positioning of the fins, etc., should also avoid interference with where a user may normally grip the implement during use. The first fin, etc., may also be closer to the first longitudinal end of the writing implement than to a mid-plane, midpoint or center point of the implement, and/or the second fin, etc., may be closer to the second longitudinal end of the writing implement than to the mid-plane, midpoint or center point of the implement. Additional fins, etc., may also be present to provide additional function(s) and/or for styling reasons. The fins, etc., of multiple writing implements intended for use together may have the same positioning and may become aligned when stacked together inside a device of the present invention, which may also serve to minimize points of contact between the stacked implements to increase the irradiated surface area of the implements.

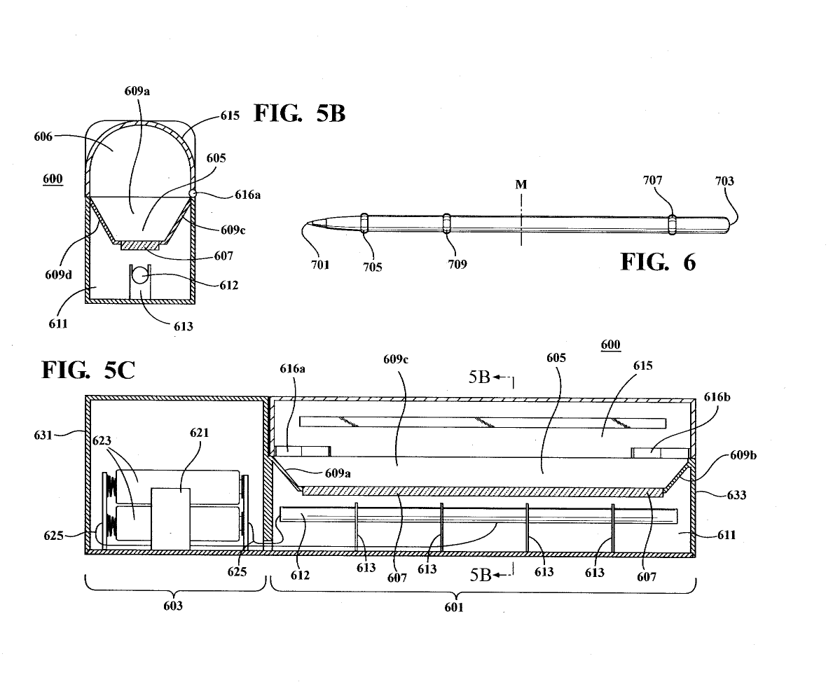

According to one embodiment of the present invention as shown in FIG. 6, a writing implement 700 having three rings 705, 707, 709 is shown. The writing implement 700 may be further described as having a first longitudinal end or writing end 701 and a second longitudinal end 703 at the opposite end of the implement 700. A first separating ring 705 is shown near the first writing end 701 of the implement 700, and a second separating ring 707 is shown near the second end 703 of the implement 700. The first separating ring 705 is further shown closer to the first writing end 701 of the implement 700 than to a longitudinal mid-plane (M) or midpoint of the implement 700, and the second separating ring 707 is shown closer to the second end 703 of the implement 700 than to the mid-plane (M) or midpoint of the implement 700. A third separating ring 709 is further shown between the first end 701 and the mid-plane (M) of the implement 700 but closer to the mid-plane (M) than the first end 701 in comparison to the first separating ring 705 of the implement 700. A handle or gripping portion on the outer side(s) or surface(s) of the writing implement may be defined between the first and third rings 705, 709.

According to another broad aspect of the present invention, methods are provided for disinfecting, sanitizing, etc., one or more writing implements using high energy rays or UV light, such as by operating or using any of the devices of the present invention). In a first step, one or more writing implement(s) may be placed, inserted, deposited, etc., into a chamber of a device of the present invention. The writing implement(s) may then be exposed to a high energy rays or UV light from one or more high energy sources or lamps to disinfect or sterilize the writing implement(s). A writing implement may then be ejected, dropped, etc., out of the device for access by a user. Such ejecting, etc., may occur by movement of a holder from a first position to a second position to release the writing implement. The holder may then return to the first position, such as to retain any writing implement(s) still inside the chamber. Such ejecting, etc., may also occur in response to a user input. These steps may occur in any of the ways described above in connection with the various devices, including those in FIGS. 1-4.

Methods of operating or using a cradle-type device of the present invention are also provided. According to these embodiments, a writing implement(s) may be placed, set, put, inserted, etc., into, on or in a receiving portion of the device. The writing implement(s) may then be exposed to a high energy rays or UV light from one or more high energy sources or lamps to disinfect or sterilize the writing implement(s). Prior to the exposure step, a cover or lid may be closed, shut, or placed on top of the receiving portion to form an enclosed portion or space within the device above the receiving portion. Such enclosed space may be around or surrounding the writing implement(s) in the device. The writing implement(s) may then be removed for use by opening, lifting, removing, etc., the cover or lid to access the writing implement(s). These steps may occur in any of the ways described above in connection with the various devices, including the device in FIG. 5.

While the present invention has been disclosed with reference to certain embodiments, it will be apparent that modifications and variations are possible without departing from the spirit and scope of the invention as defined herein. Furthermore, it should be appreciated that all examples in the present disclosure, while illustrating embodiments of the invention, are provided as non-limiting examples and are, therefore, not to be taken as limiting the various aspects so illustrated. The present invention is intended to have the full scope of the invention as defined herein and equivalents thereof. Accordingly, the drawings and description are to be regarded as illustrative and not as restrictive.

What is claimed is:

1. A device for disinfecting one or more writing implements comprising:
two or more portions surrounding an inner chamber, the two or more portions comprising a front portion and a back portion; and
at least one high energy source or bulb for directing disinfecting light toward the inner chamber of the device, each high energy source or bulb being positioned within one of the portions of the device,
wherein the inner chamber has a top opening and a bottom opening, and
wherein the horizontal cross-sectional shape of the inner chamber has an elongated width dimension and a depth dimension, the width dimension of the inner chamber being at least four times greater than the depth dimension of the inner chamber.

2. The device of claim 1, wherein the two or more portions of the device further comprise a left side portion and a right side portion.

3. The device of claim 1, wherein the at least one high energy source or bulb is an ultraviolet (UV) bulb for producing disinfecting UV light.

4. The device of claim 1, wherein the front portion and the back portion of the device each comprise an outer wall.

5. The device of claim 4, wherein the front portion of the device further comprises a front compartment between an inner wall of the front portion and the outer wall of the front portion.

6. The device of claim 5, wherein one or more high energy sources or bulbs are present in the front compartment of the device.

7. The device of claim 6, wherein the inner wall of the front portion of the device is transparent to allow the disinfecting light from the one or more high energy sources or bulbs in the front compartment to reach the inner chamber of the device.

8. The device of claim 7, wherein the back portion of the device further comprises a back compartment between an inner wall of the back portion and the outer wall of the back portion.

9. The device of claim 8, wherein one or more high energy sources or bulbs are present in the back compartment of the device.

10. The device of claim 6, wherein the inner side or surface of the outer wall of the front portion of the device is reflective to reflect the disinfecting light.

11. The device of claim 3, wherein the back portion of the device further comprises a back compartment between an inner wall of the back portion and the outer wall of the back portion.

12. The device of claim 11, wherein one or more high energy sources or bulbs are present in the back compartment of the device.

13. The device of claim 12, wherein the inner wall of the back portion of the device is transparent to allow the disinfecting light from the one or more high energy sources or bulbs in the back compartment to reach the inner chamber of the device.

14. The device of claim 12, wherein the inner side or surface of the outer wall of the back portion of the device is reflective to reflect the disinfecting light.

15. The device of claim 1, further comprising:
a holder, the holder extending into the inner chamber of the device; and
an actuator, the actuator being coupled to the holder and causing the holder to move from a first holding position in contact with a writing implement inside the inner chamber to a second releasing position to release the writing implement to fall out of the device through the bottom opening of the chamber of the device.

16. The device of claim 15, wherein the holder moves reversibly between the first position and the second position.

17. The device of claim 15, wherein the holder moves between the first position and the second position in a non-reversible cycle.

18. The device of claim 15, further comprising:
a sensor, the sensor receiving an input from a user and communicating the input to cause release of a writing implement by the holder.

19. The device of claim 18, further comprising:
a controller, the controller controlling the operation of the holder for causing the release of a writing implement from the inner chamber of the device in response to an input received by the sensor and communicated to the controller.

20. The device of claim 15, further comprising:
a controller, the controller controlling the operation of the holder for causing the release of a writing implement from the inner chamber of the device.

21. The device of claim 15, further comprising:
a controller, the controller controlling the timing of operation of the at least one high energy source or bulb for causing high energy irradiation of the one or more writing implements inside the inner chamber of the device.

22. The device of claim 1, further comprising:
a shield, the shield being located at or near the top opening of the chamber.

23. The device of claim 1, further comprising:
a tray, the tray being located underneath the bottom opening of the chamber for receiving a released writing implement form within the inner chamber of the device.

24. The device of claim 1, further comprising:
a stand, the stand connected to the device and configured to support the device when the stand is placed on a support surface.

25. The device of claim 1, further comprising:
a support, the support connected to the device and configured to attach to another device.

26. A device for disinfecting a writing implement comprising:
a cradle portion having a receiving area, the receiving area having a length dimension and a depth dimension, the length dimension of the receiving area being at least four times greater than the depth dimension of the receiving area;
at least one high energy source or bulb for directing disinfecting light toward the receiving area of the device; and
a cover or lid, the cover or lid enclosing the space above the receiving area of the device when the cover or lid is in a closed position relative to the cradle portion of the device.

27. The device of claim 26, wherein the cradle portion further comprises a lower compartment, the at least one high energy source or bulb being positioned within the lower compartment of the device, wherein the lower compartment is at least partially separated from the receiving area of the device by a transparent window.

28. The device of claim 27, further comprising:
a power source; and
a controller, the controller being in electrical communication with the power source and the at least one high energy source or bulb, the controller determining when the at least one high energy source or bulb is turned on.

29. The device of claim 28, wherein the power source and the controller are located within a housed portion of the device.

30. The device of claim 26, wherein the receiving area comprises a recessed cavity.

31. A writing implement having elongated dimensions with a first longitudinal end and a second longitudinal, the first and second longitudinal ends being on opposite ends of the writing implement, the writing implement comprising:
a plurality of outer fins including a first separating fin and a second separating fin, the first separating fin being closer to the first longitudinal end of the writing implement and the second separating fin being closer to the second longitudinal end of the writing implement.

32. The writing implement of claim 31, wherein the first separating fin is closer to the first longitudinal end than to a midpoint of the writing implement, and wherein the second separating fin is closer to the second longitudinal end than to the midpoint of the writing implement.

33. The writing implement of claim 31, wherein the first longitudinal end is the writing end.
